(19) United States
(12) Patent Application Publication
(10) Pub. No.: US 2008/0119884 A1
(43) Pub. Date: May 22, 2008

(54) Single Puncture Lancing Fixture with Depth Adjustment and Control of Contact Force

(76) Inventors: Bruce A. Flora, Elkhart, IN (US); Donald R. Hesser, Decatur, MI (US); Allen J. Brenneman, Goshen, IN (US); George V. Siddons, Elkhart, IN (US)

Correspondence Address: NIXON PEABODY LLP, 161 N. CLARK STREET, 48TH FLOOR, CHICAGO, IL, 60601, US

(21) Appl. No.: 11/661,226

(22) PCT Filed: Sep. 8, 2005
(86) PCT No.: PCT/US05/31766

§ 371 Date: Sep. 27, 2007

Related U.S. Application Data
(60) Provisional application No. 60/608,491, filed on Sep. 9, 2004.

(51) Int. Cl. A61B 5/151

(52) U.S. Cl. 606/182; 606/181

(57) ABSTRACT

A lancing mechanism fixture for puncturing skin with a lancet having a penetration end being moveable in a direction substantially parallel to the longitudinal axis of the lancet, the penetration end being moveable from a first position to a second position during a forward stroke, the penetration end being movable from the second position back to the first position during a return stroke. The motion of the lancet is controlled by a cam mechanism including a slot cam and a cam follower connected to a lancet. A drive member applies a linear force to the slot cam whose linear motion and slot path shape forces the lancet to move from a first position to a second position and back to the first position.

### CROSS-REFERENCE TO RELATED APPLICATIONS

This application claims priority to Application No. 60/608,491, filed Sep. 9, 2004.

### FIELD OF THE INVENTION

The present invention relates generally to blood monitoring devices, and more specifically, to a lancing mechanism for puncturing a user's skin to obtain a blood sample for analysis.

### BACKGROUND OF THE INVENTION

It is often necessary to quickly obtain a sample of blood and perform an analysis of the blood sample. Preferably, the obtaining of blood is as painless as possible. One example of a need for painlessly obtaining a sample of blood is in connection with a blood glucose monitoring system where a user must frequently use the system to monitor the user's blood glucose level.

Those who have irregular blood glucose concentration levels self-monitor their blood glucose concentration level. An irregular blood glucose level can be brought on by a variety of reasons including illness such as diabetes. The purpose of monitoring the blood glucose concentration level is to determine the blood glucose concentration level and then to take corrective action, based upon whether the level is too high or too low, to bring the level back within a normal range. The failure to take corrective action can have serious implications. When blood glucose levels drop too low—a condition known as hypoglycemia—a person can become nervous, shaky, and confused, which may result in a person passing out. A person can also become very ill if their blood glucose level becomes too high—a condition known as hyperglycemia. Both conditions, hypoglycemia and hyperglycemia, are both potentially life-threatening emergencies.

One method of monitoring a person's blood glucose level is with a portable, hand-held blood glucose testing device. The portable nature of these devices enables the users to conveniently test their blood glucose levels wherever the user may be. To check the blood glucose level, a drop of blood is obtained from the fingertip using a lancing device. The lancing device contains a lancet to puncture the skin. Once the requisite amount of blood is produce on the fingertip, the blood is harvested using the blood glucose testing device and the glucose concentration is determined.

Despite significant improvements in the lancing mechanism field, puncture depth variations remain a problem. Many prior art lancing devices implement a spring coupled to the actual lancet to move the lancet to its penetration depth. The lancet is drawn back to compress the spring. When released, the spring extends, thereby forwardly propelling the lancet to its penetration depth. One problem associated with some prior art lancing devices is that the penetration depth of those lances is dependant on a spring constant, which is a measure of the spring's stiffness. The mechanical qualities of a spring, including the stiffness, tend to degrade over time with use. Similarly, spring mountings are subject to “creep” or deformation if overstressed. Accordingly, over time, the penetration depth of many prior art lances may lessen. When the penetration depth of a lancet lessens over time, the lancet may not produce a laceration deep enough to draw the requisite volume of blood necessary for proper blood glucose analysis. An insufficient lancing can result in an erroneous analysis if the user does not recognize that the lancing has not produced the requisite blood amount or volume for analysis. Or, if the user does recognize an insufficient lancing has occurred, the user must re-lance, resulting in another laceration in the user's skin and more pain. The user will eventually have to replace a lancet that has degraded over time.

A similar problem associated with many of the prior art lancing devices is that when the spring forwardly advances the lancet to its penetration depth, the spring extends past its static length. When this occurs, the spring then retracts the lancet. Due to the oscillatory nature of the spring, however, the lancet is retracted past its static length. Thus, the lancet continues to oscillate, causing the lancet penetration end to enter the laceration created in the user's skin several times. Put another way, with each actuation of a spring constant dependant device, a user's skin is lanced several times, which results in a larger laceration. A larger laceration in the user' skin, in turn, results in more pain for the user and a longer time for the laceration to heal. To mitigate this oscillating effect, some prior art devices have employed the use of dampers and internal stops. The performance of these features, however, is unpredictable due to a variety of factors, such as component tolerances.

Another problem associated with many of the prior art lancing devices is that they do not allow the user to accurately control puncture depth by precisely adjusting the clearance between the lancet needle tip and the skin. While some prior art devices use adjustable endcaps to vary the distance between the skin and the lancet needle tip, puncture depth may remain erratic because of (a) the variation in disposable lancet needle lengths, and (b) the use of opaque endcaps that prevent visual confirmation of the distance between the skin and the needle tip by the user. Further compounding this puncture depth problem is that, because skin is elastic, variation in the force applied to the endcap results in different degrees of stretching or bulging of the skin in relation to the endcap hole size, which will vary the clearance between the skin and the lancet needle tip. No prior art lancing device controls the amount of force applied to the endcap.

Accordingly, there exists a need for a lancing mechanism that precisely moves a lancet a known distance, allows for visual adjustment of puncture depth, and controls contact force.

### SUMMARY OF THE INVENTION

A lancing mechanism for puncturing skin is provided. The lancing mechanism comprises a lancet having a penetration end that is adapted to puncture skin. The penetration end of the lancet is movable from a first position to a second position during a forward stroke and moveable from the second position back to the first position during a return stroke.

The motion of the lancet is controlled by a cam mechanism including a slot cam and a cam follower. The lancet is connected to a cam follower, which is engaged to a moveable slot cam. A drive member applies a linear force to the slot cam whose linear motion and slot path shape forces the lancet to move from a first position to a second position and back to the first position.

### BRIEF DESCRIPTION OF THE DRAWINGS

FIG. 1a is a top view of a lancing mechanism shown in a pre-lancing position according to one embodiment of the present invention.

FIG. 1b is a is a perspective view of a lancing mechanism shown in a pre-lancing position according to one embodiment of the present invention.

FIG. 2a is a top view of a lancing mechanism shown at the approximate mid-stroke position according to one embodiment of the present invention.

FIG. 2b is a perspective view of a lancing mechanism shown at the approximate mid-stroke position according to one embodiment of the present invention.

FIG. 3a is a top view of a lancing mechanism shown in a post-lancing position according to one embodiment of the present invention.

FIG. 3b is a perspective view of a lancing mechanism shown in a post-lancing position according to one embodiment of the present invention.

### DETAILED DESCRIPTION OF THE INVENTION

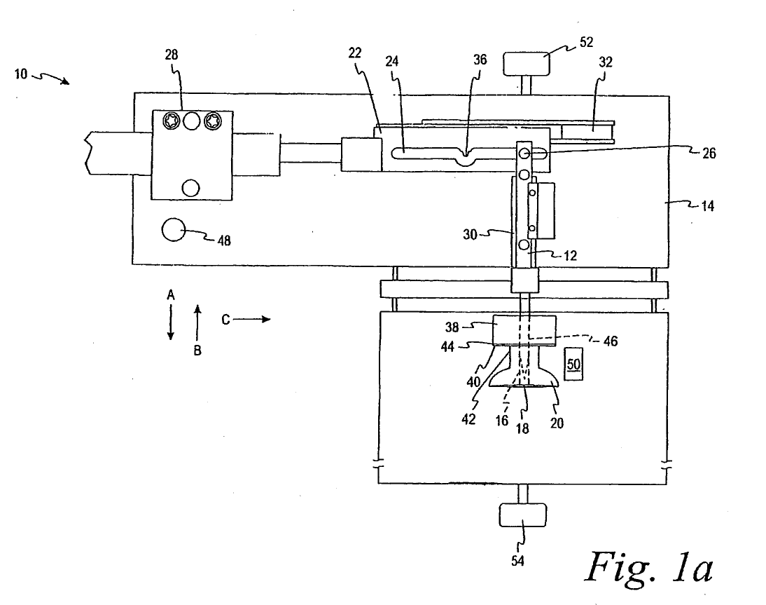

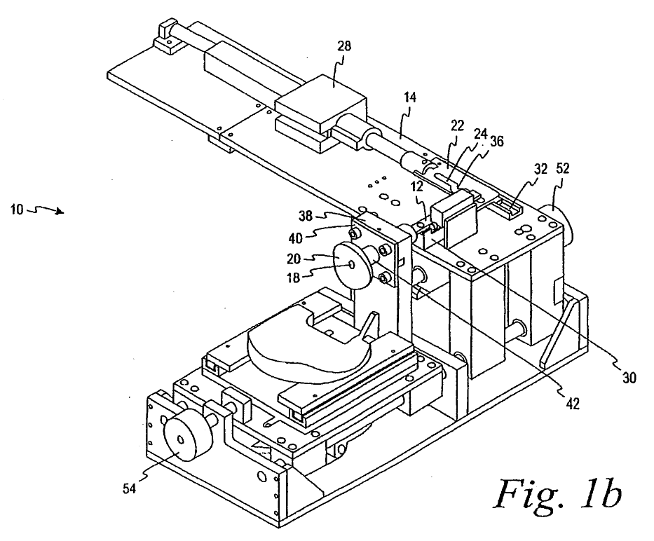

Referring to FIGS. 1a and 1b, a lancing mechanism 10 of the present invention will be described in greater detail. The lancing mechanism 10 includes a lancet 12 on a fixture 14. The lancet 12 has a sharp penetration end 16 that is adapted to puncture skin to obtain a blood drop for analysis. To obtain a blood sample, the penetration end 16 of the lancet 12 extends through a hole 18 formed in an endcap 20 to puncture the user's skin. After making the initial laceration in the user's skin, the lancet 12 returns back through the hole 18 in the endcap 20.

The lancing mechanism 10 desirably fires the lancet 12 without experiencing the oscillations that result in a larger laceration. During a stroke, the penetration end 16 of the lancet 12 is guided through the endcap 20 to its penetration depth and back by the movement of a slot cam 22. The slot cam 22 in this embodiment has a generally “v” or “u” shaped or curved slot path 24 formed therein that forces a cam follower 26 attached to lancet 12 to trace the slot path 24. The lancet 12 is moved a known distance during a stroke with a cam follower 26 engaged with the slot cam 22. The stroke distance is determined by the slot path 24 of the slot cam 22 and not on a spring constant and the extension of that spring past the static length of the spring.

A linear motor 28 in this embodiment provides the force necessary to move the penetration end 16 of the lancet 12 through the user's skin to the desired penetration depth. The linear motor 28 forces to the slot cam 22 to move in a direction parallel to its longitudinal axis. The linear movement of the slot cam 22 guides the cam follower 26 attached to the lancet 12 along the slot path 24, forcing the attached lancet 12 to move in a direction substantially perpendicular to the direction of the slot cam 22. The linear motor 28 may be activated by a trigger 48. When activated, the trigger starts the linear motor 28, which results in moving the slot cam 22 and firing the lancing mechanism 10.

In an alternative embodiment of the present invention, the linear motor 28 may be replaced by a spring drive mechanism. In this alternative embodiment, the extension of a compressed spring connected to the slot cam 22 will provide the force necessary to move the slot cam 22 in a linear direction, resulting in the firing of the lancing mechanism 10.

The lancet 12 that is positioned in a pre-firing position (FIG. 1a) is movable in the directions indicated by arrow A and arrow B during the stroke depending on where the lancet 12 and cam follower 26 are positioned on the slot path 24. The fixture 14 includes a first channel 30 that constrains the movement of the lancet 12. The first channel 30 is substantially parallel to the longitudinal axis of the lancet 12.

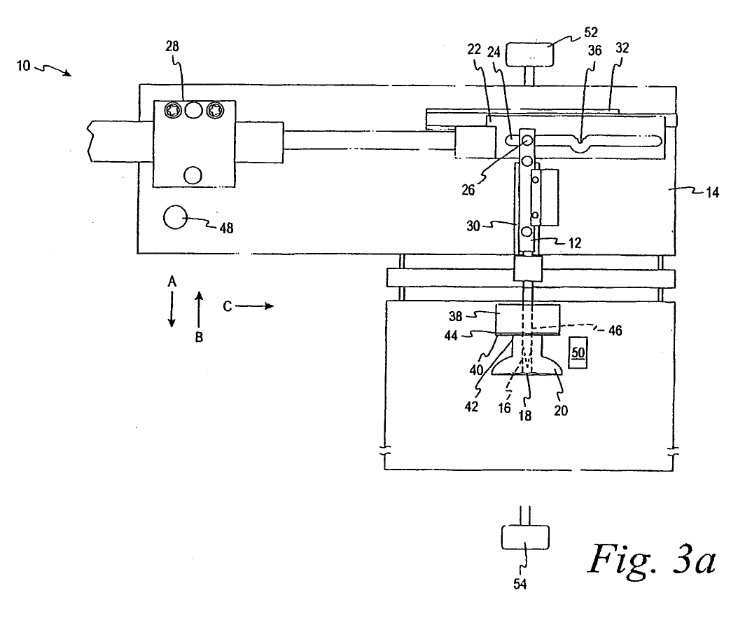

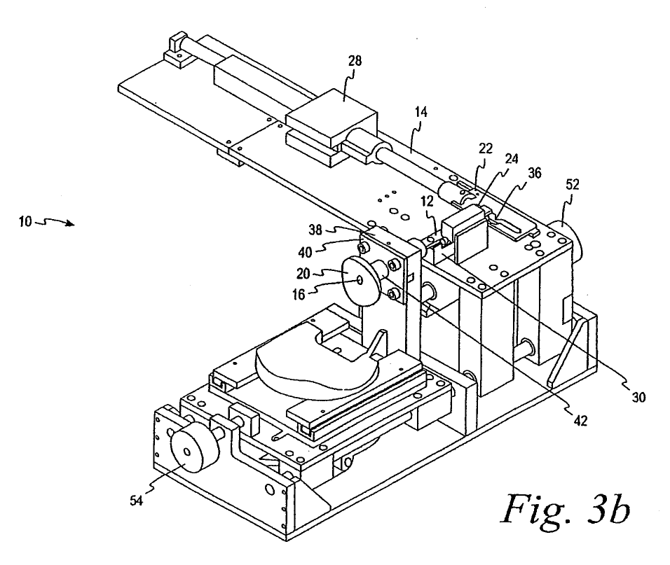

Referring still to FIGS. 1a and 1b, the fixture 14 also includes a second channel 32 that constrains the movement of the slot cam 22. The slot cam 22 and the second channel 32 are disposed on the fixture 14 substantially perpendicular to the lancet 12. The slot cam 22 moves in the direction indicated by the arrow C from a pre-firing position (FIG. 1a) to a post-firing position (FIG. 3a). The movement of the slot cam 22 from the pre-firing position to the post-firing position generates the movement of the lancet 12 from a first position to a second position back to the first position. The slot cam 22 has a generally “v” or “u” shaped curve 36 disposed in the slot path 24. The combination of the shape of the slot path 24 and the linear motion of the slot cam 22 guides the lancet 12 in a direction towards the endcap 20 and perpendicular to the direction of travel of the slot cam 22.

The fixture 14 contains an endcap mounting plate 38. The endcap mounting plate 38 is positioned such that the lancet passes through a hole 46 formed in the endcap mounting plate 38. An annular load cell 40 is mounted onto the back side 44 of the endcap mounting plate 38. The base 42 of the endcap 20 is mounted on the annular load cell 40. The endcap 20 and annular load cell 40 are positioned along the lancet's 12 travel axis such that, during a stroke, the penetration end 16 of the lancet 12 extends through the hole 18 formed in the endcap 20. The annular load cell 40 electronically registers the force applied to the annular load cell 40 from the endcap 20 when force is applied to the endcap 20, and starts the linear motor 28 when a predetermined force is applied.

In an alternative embodiment of the present invention, the force registering, electronic annular load cell 40 may be replaced by a mechanical spring loaded mechanism. This spring loaded mechanism includes spring with a pre-set stiffness, such that the amount of force applied to the endcap 20 is determined based on the distance that the spring is compressed. When compressed a predetermined distance, the spring loaded mechanism will activate the linear motor 28.

The fixture 14 contains a high-speed video system 50 to (a) observe and record the events occurring within the endcap 20, and (b) measure and facilitate adjustment of puncture depth of the lancet 12 by verifying the clearance between the surface of the skin and the penetration end 16. The video system 50 may be positioned adjacent to the endcap 20 to observe the penetration end 16 of the lancet 12 pass through the hole 18 in the endcap 20 and into the skin surface. It is contemplated that the endcap 20 be transparent and/or the endcap 20 form a slot or a window therein, through which the video system may observe the events occurring within the endcap 20.

In an alternative embodiment, the high-speed video system 50 is replaced by an endcap with a viewing lens and suitable measurement markings. In this alternative embodiment, the user can physically view and verify the clearance between the surface of the skin and the penetration through the viewing lens and adjust the puncture depth of the lancet 12 as desired.

Referring now to FIG. 1b, the fixture 14 contains a penetration end adjustment mechanism 52. It is contemplated that the penetration end adjustment mechanism 52 includes a threaded knob assembly that causes the portion of the fixture 14 upon which the lancet 12 is disposed to move parallel to the lancet's travel axis. The penetration end adjustment mechanism 52 allows adjustment of the position of the penetration end 16 relative to the skin surface in the endcap 20. The fixture 14 also contains an endcap adjustment mechanism 54. It is contemplated that the endcap adjustment member 54 includes a threaded knob assembly that causes the portion of the fixture 14 that containing the endcap mounting plate 38 to move parallel to the lancet's 12 travel axis. The endcap adjustment mechanism 54 allows adjustment of the position of the endcap 20 relative to the position of the penetration end 16.

In an alternative embodiment, the endcap adjustment mechanism 54 and threaded knob assembly may be replaced by the use of an adjustable endcap that includes a mechanism for adjusting the clearance between the penetration end 16 and the skin surface. An example of such an adjustable endcap is found in U.S. Pat. No. 5,916,230, which is incorporated herein by reference.

The operation of the lancing mechanism will now be described starting with FIGS. 1a and 1b. To lance a user's skin, a user presses their skin against the endcap 20 along the lancet travel axis. The skin will typically be the skin of the user's finger or hand. It is contemplated, however, that alternative sites may be used. In FIGS. 1a and 1b, the lancing mechanism 10 is shown in the pre-firing position with the lancet 12 in a first position. As the user applies a force to the endcap 10, the annular load cell 40 measures and registers the amount of force applied. The user continues to apply a force to the endcap 20 until a pre-determined amount of force is achieved and registered by the annular load cell 40.

In one embodiment of the present invention, when the pre-determined (threshold) amount of force is achieved, an indicator lamp (not shown) is lit, signaling to the user that the desired pressure is being applied. In another embodiment of the present invention, a visible force gauge (not shown) displays to the user the amount of force being applied to the endcap 20. While maintaining the predetermined amount of force against the endcap 20, the user adjusts the clearance between the skin surface in the hole 18 formed in the endcap 20 and the penetration end 16 of the lancet 12 to set a precise puncture depth. Adjustment of the puncture depth is achieved using the lancet adjustment member 52 and the endcap adjustment member 54. The pre-firing position of the penetration end 16 of the lancet 12 relative to the skin surface may be moved in both directions indicated by arrow A and arrow B. The pre-firing position of the endcap 20 relative to the penetration end 16 of the lancet 12 may also be moved in directions indicated by arrow A and arrow B.

By using a high-speed video controlled depth adjustment system 50, for example, precise adjustment to the penetration end 16 and skin surface positions may be controlled. The high-speed video system 50 shows the user an image of the inside of the endcap 20. To facilitate verification and precise adjustment of the clearance between the skin surface and the penetration end 16, the video system 50 superimposes pre-measured measurement markings onto the image of the inside of the endcap 20. In an alternative embodiment, the penetration end 16 and skin surface positions may be verified by using measurement markings visible on or adjacent to a transparent endcap or a viewing lens in an opaque endcap.

After the clearance between the skin surface and the penetration end 16 is adjusted while a pre-determined force is applied to the endcap 20, the user then arms the lancing mechanism trigger 48. Once armed, the trigger 48, when activated, starts the linear motor 28 and thus fires the lancing mechanism 10. Thus, in one embodiment, the lancet will be fired when (a) the lancing mechanism 10 is armed, and (b) the user applies the pre-determined amount of force to the endcap 20 used to set the puncture depth. If the proper amount of force is applied, then the linear motor 28 is started, resulting in the lancing mechanism 10 being fired.

When started, the linear motor 28 rapidly accelerates the slot cam 22 in the direction indicated by the arrow C in FIG. 1a. The linear movement of the slot cam 22 in the direction of arrow C guides the cam follower 26 along the slot path 24. In turn, the linear movement of the slot cam 22, along with the “v” or “u” shape 36 of the slot path 24 forces the lancet 12 attached to the cam follower 26 to travel in the linear direction indicated by the arrow A (forward stroke).

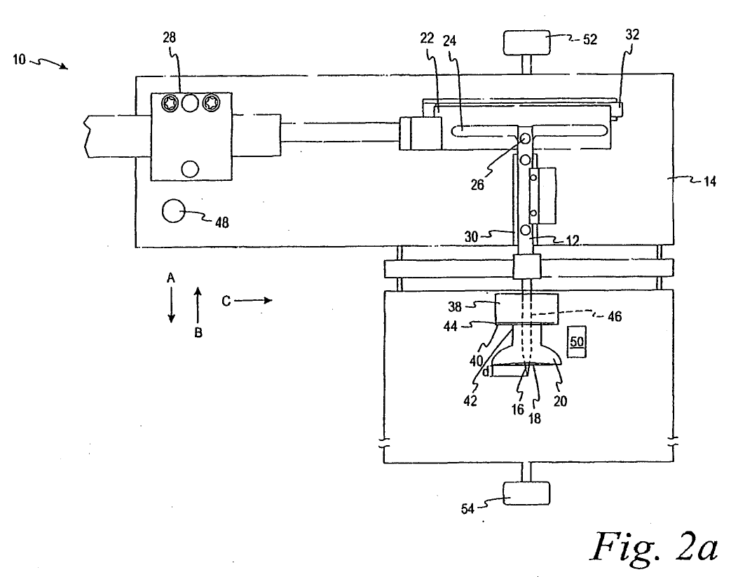

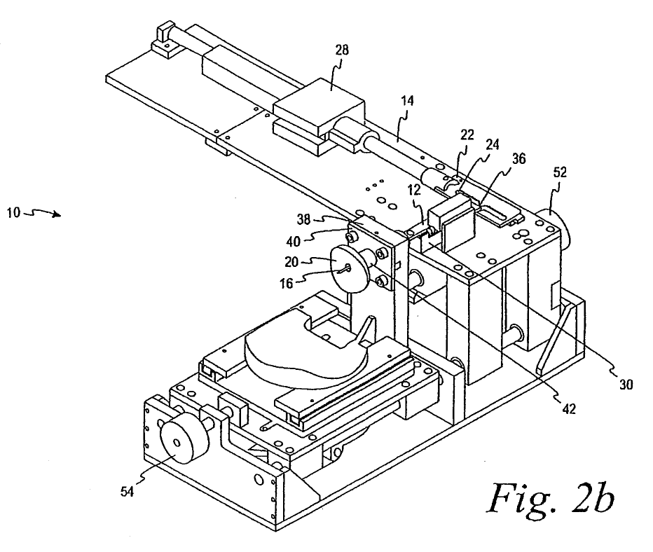

Turning now to FIGS. 2a and 2b, the linear motor 28 has caused the slot cam 22 to move approximately one-half the distance of its allowed travel, such that the “v” or “u” shape 36 of the slot path 24 has guided the cam follower 26 and attached lancet 12 to the bottom of the “v” or “u” shape 36 of the slot path 24. When in this second position, the penetration end 16 has been moved a distance sufficient for the penetration end 16 to extend beyond the hole 18 formed in the endcap 20 a distance d equivalent to the penetration depth. The distance d is dependant on the size and shape of the slot path 24 along with the pre-firing clearance between the skin surface and penetration end. As shown in FIGS. 2a and 2b, the lancet 12 stops moving in the direction indicated by arrow A when the attached cam follower 26 reaches the bottom of the “v” or “u” shape 36 of the slot path 24.

Turning now to FIGS. 3a and 3b, the linear motor 28 has caused the slot cam 22 to move the entire distance allowed by the linear motor and the second channel (or a full stroke). As the slot cam 22 continues to travel in the direction indicated by arrow C, the slot path 24 continues to guide the engaged cam follower 26 and lancet along the “v” or “u” shape 36 of the slot path 24. As discussed above, once the cam follower 26 reaches the bottom of the “v” or “u” shape 36 of the slot path 24 (FIGS. 2a, 2b), the cam follower 26 is no longer being forced in the direction indicated by arrow A. The continued linear movement of the slot cam 22 in the direction of arrow C forces the lancet 12 attached to the cam follower 26 to follow the slot path 24, which results in the lance traveling in the direction indicated by arrow B to the position shown in FIGS. 3a and 3b (return stroke). When in the position shown in FIGS. 3a and 3b, the penetration end 16 is retracted away from the skin and back through the hole 18 formed in the endcap 20 a distance equivalent to the distance traveled during the forward stroke. The penetration end 16 returns to the same (first) position as before the lancing mechanism 10 was fired (return stroke). Thus, a firing of the lancing mechanism 10 results in only one forward stroke and one return stroke of the lancet 12. The lancet 12 does not oscillate because the linear movement of the lancet 12 is constrained by the fixed shape of the slot path 24, which prevents any further movement of the lancet 12 and penetration end 16 in the direction of arrow A. The use of the slot cam 22 results in the travel distance of the lancet remaining fixed over the life of the lancing mechanism 10, thereby allowing the user to consistently and precisely set the puncture depth.

In the present invention, the forward stroke of the lancet 12 is dependant on the size and shape of the slot path 24 of the slot cam 22 along with the clearance between the surface of the skin and the penetration end 16. It is contemplated that the shape of the slot path 24 may be altered so as to change the fixed travel distance of the lancet 12. It is further contemplated that the shape of the slot path 24 may be altered so as to vary the speed at which the lancet 12 travels.

### EMBODIMENT A

A lancing mechanism fixture for puncturing skin comprising:

a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;

a cam mechanism including a slot cam and a cam follower, the cam follower connected to the lancet, the cam follower engaged to the slot cam such that the longitudinal axis of the slot cam is generally perpendicular to the longitudinal axis of the lancet, the slot cam being moveable along its longitudinal axis to move the cam follower and connected lancet a fixed distance; and a drive member connected to the slot cam, the drive member adapted to assist in moving the slot cam.

### EMBODIMENT B

The lancing mechanism of Embodiment A further comprising an endcap located along the lancet's travel axis, the endcap positioning a surface of the skin a distance from the penetration end, the endcap including a hole therethrough in which the penetration end passes to puncture the skin surface.

### EMBODIMENT C

The lancing mechanism of Embodiment B further including a force registering member connected to the endcap that registers the amount of force applied to the endcap.

### EMBODIMENT D

The lancing mechanism of Embodiment C wherein the force registering member is an annular load cell.

### EMBODIMENT E

The lancing mechanism of Embodiment C where in the force registering member is a spring loaded mechanism.

### EMBODIMENT F

The lancing mechanism of Embodiment C wherein the force registering member activates the drive member when a predetermined amount of force is applied to the endcap.

### EMBODIMENT G

The lancing mechanism of Embodiment B further including a penetration end adjustment mechanism and an endcap adjustment mechanism for adjusting the puncture depth of the lancet.

### EMBODIMENT H

The lancing mechanism of Embodiment G further including a high speed video system.

### EMBODIMENT I

The lancing mechanism of Embodiment G wherein the endcap is generally transparent with measurement markings.

### EMBODIMENT J

The lancing mechanism of Embodiment G wherein the endcap includes a viewing lens and measurement markings.

### EMBODIMENT K

The lancing mechanism of Embodiment A wherein the drive member is a linear induction motor.

### EMBODIMENT L

The lancing mechanism of Embodiment A wherein the drive member is a spring drive mechanism.

### EMBODIMENT M

The lancing mechanism of Embodiment A wherein the longitudinal axis of the slot cam is substantially perpendicular to the longitudinal axis of the lancet.

### EMBODIMENT N

The lancing mechanism of Embodiment M wherein the longitudinal axis of the slot cam is perpendicular to the longitudinal axis of the lancet.

### EMBODIMENT O

A lancing mechanism fixture for puncturing skin comprising:

a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;

a cam mechanism including a slot cam and a cam follower, the cam follower connected to the lancet, the cam follower engaged to the slot cam such that the longitudinal axis of the slot cam is substantially perpendicular to the longitudinal axis of the lancet, the slot cam being moveable along its longitudinal axis to move the cam follower and connected lancet a fixed distance;

a drive member connected to the slot cam, the drive member adapted to assist in moving the slot cam; and

an endcap located along the lancet's travel axis, the endcap positioning a surface of the skin a distance from the penetration end, the endcap comprising a hole therethrough in which the penetration end passes to puncture the skin surface.

### EMBODIMENT P

The lancing mechanism of Embodiment O further including a force registering member connected to the endcap that registers the amount of force applied to the endcap.

### EMBODIMENT Q

The lancing mechanism of Embodiment P wherein the force registering member is an annular load cell.

### EMBODIMENT R

The lancing mechanism of Embodiment P where in the force registering member is a spring loaded mechanism.

### EMBODIMENT S

The lancing mechanism of Embodiment P wherein the force registering member activates the drive member when a predetermined amount of force is applied to the endcap.

### EMBODIMENT T

The lancing mechanism of Embodiment O further including a penetration end adjustment mechanism and an endcap adjustment mechanism for adjusting the puncture depth of the lancet.

### EMBODIMENT U

The lancing mechanism of Embodiment T further including a high speed video system.

### EMBODIMENT V

The lancing mechanism of Embodiment T wherein the endcap is generally transparent with measurement markings.

### EMBODIMENT W

The lancing mechanism of Embodiment T wherein the endcap includes a viewing lens and measurement markings.

### EMBODIMENT X

The lancing mechanism of Embodiment O, wherein the drive member comprises a linear induction motor.

### EMBODIMENT Y

The lancing mechanism of Embodiment O, wherein said drive member comprises a spring drive mechanism.

### EMBODIMENT Z

The lancing mechanism of Embodiment O, wherein the longitudinal axis of the slot cam is perpendicular to the longitudinal axis of the lancet.

### EMBODIMENT AA

A lancing mechanism fixture for puncturing skin comprising:

a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;

a cam mechanism including a slot cam and a cam follower, the cam follower connected to the lancet, the cam follower engaged to the slot cam such that the longitudinal axis of the slot cam is generally perpendicular to the longitudinal axis of the lancet, the slot cam being moveable along its longitudinal axis to move the cam follower and connected lancet a fixed distance;

a spring device connected to the slot cam, the spring device adapted to assist in moving the slot cam a fixed distance; an endcap, located along the lancet's travel axis positioning the skin surface a distance from the penetration end, the endcap comprising a hole through which the penetration end passes to puncture the skin;

a force registering member connected to the endcap that registers the amount of force applied to the endcap, the force registering member activating the drive member when a predetermined amount of force is applied to the endcap; and

a penetration end adjustment mechanism and an endcap adjustment mechanism for adjusting the puncture depth of the penetration end of the lancet.

Process BB

A method for puncturing skin to obtain a blood sample with a lancing mechanism, the lancing mechanism including a slot cam, lancet, endcap, drive member, and trigger, the method comprising the acts of:

pressing the user's skin against the endcap connected to a force registering member;

determining the force applied to the endcap; and

initiating the drive member if a predetermined amount of force is applied to the endcap, the drive member causing the slot cam to move in a linear direction, the linear movement of the slot cam causing the lancet to move from a first position to a second position and back to the first position, resulting in a single puncture in the user's skin.

Process CC

The method of Process BB, further including the act of adjusting the position of the penetration end and the endcap to ensure the desired clearance between the skin surface and the penetration end.

While the present invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined in the appended claims.

1. A lancing mechanism fixture for puncturing skin comprising:
a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;
a cam mechanism including a slot cam and a cam follower, the cam follower connected to the lancet, the cam follower engaged to the slot cam such that the longitudinal axis of the slot cam is generally perpendicular to the longitudinal axis of the lancet, the slot cam being moveable along its longitudinal axis to move the cam follower and connected lancet a fixed distance; and
a drive member connected to the slot cam, the drive member adapted to assist in moving the slot cam.

2. The lancing mechanism of claim 1, further comprising an endcap located along the lancet's travel axis, the endcap positioning a surface of the skin a distance from the penetration end, the endcap including a hole therethrough in which the penetration end passes to puncture the skin surface.

3. The lancing mechanism of claim 2, further including a force registering member connected to the endcap that registers the amount of force applied to the endcap.

4. The lancing mechanism of claim 3, wherein the force registering member is an annular load cell.

5. The lancing mechanism of claim 3, where in the force registering member is a spring loaded mechanism.

6. The lancing mechanism of claim 3, wherein the force registering member activates the drive member when a predetermined amount of force is applied to the endcap.

7. The lancing mechanism of claim 2, further including a penetration end adjustment mechanism and an endcap adjustment mechanism for adjusting the puncture depth of the lancet.

8. The lancing mechanism of claim 7, further including a high speed video system.

9. The lancing mechanism of claim 7, wherein the endcap is generally transparent with measurement markings.

10. The lancing mechanism of claim 7, wherein the endcap includes a viewing lens and measurement markings.

11. The lancing mechanism of claim 1, wherein the drive member is a linear induction motor.

12. The lancing mechanism of claim 1, wherein the drive member is a spring drive mechanism.

13. The lancing mechanism of claim 1, wherein the longitudinal axis of the slot cam is substantially perpendicular to the longitudinal axis of the lancet.

14. The lancing mechanism of claim 13, wherein the longitudinal axis of the slot cam is perpendicular to the longitudinal axis of the lancet.

15. A lancing mechanism fixture for puncturing skin comprising:
a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;
a cam mechanism including a slot cam and a cam follower, the cam follower connected to the lancet, the cam follower engaged to the slot cam such that the longitudinal axis of the slot cam is substantially perpendicular to the longitudinal axis of the lancet, the slot cam being moveable along its longitudinal axis to move the cam follower and connected lancet a fixed distance;
a drive member connected to the slot cam, the drive member adapted to assist in moving the slot cam; and
an endcap located along the lancet's travel axis, the endcap positioning a surface of the skin a distance from the penetration end, the endcap comprising a hole therethrough in which the penetration end passes to puncture the skin surface.

16. The lancing mechanism of claim 15, further including a force registering member connected to the endcap that registers the amount of force applied to the endcap.

17. The lancing mechanism of claim 16, wherein the force registering member is an annular load cell.

18. The lancing mechanism of claim 16, where in the force registering member is a spring loaded mechanism.

19. The lancing mechanism of claim 16, wherein the force registering member activates the drive member when a predetermined amount of force is applied to the endcap.

20. The lancing mechanism of claim 15, further including a penetration end adjustment mechanism and an endcap adjustment mechanism for adjusting the puncture depth of the lancet.

21. The lancing mechanism of claim 20, further including a high speed video system.

22. The lancing mechanism of claim 20, wherein the endcap is generally transparent with measurement markings.

23. The lancing mechanism of claim 20, wherein the endcap includes a viewing lens and measurement markings.

24. The lancing mechanism of claim 15, wherein the drive member comprises a linear induction motor.

25. The lancing mechanism of claim 15, wherein said drive member comprises a spring drive mechanism.

26. The lancing mechanism of claim 15, wherein the longitudinal axis of the slot cam is perpendicular to the longitudinal axis of the lancet.

27. A lancing mechanism fixture for puncturing skin comprising:
a lancet disposed on the fixture having a penetration end being adapted to puncture skin, the penetration end being moveable in a direction substantially parallel to a longitudinal axis of the lancet, the penetration end of the lancet being moveable from a first position to a second position during a forward stroke, the penetration end of the lancet being movable from the second position back to the first position during a return stroke;
a cam mechanism including a slot cam and a cam follower, the cam follower connected to the lancet, the cam follower engaged to the slot cam such that the longitudinal axis of the slot cam is generally perpendicular to the longitudinal axis of the lancet, the slot cam being moveable along its longitudinal axis to move the cam follower and connected lancet a fixed distance;
a spring device connected to the slot cam, the spring device adapted to assist in moving the slot cam a fixed distance;
an endcap, located along the lancet's travel axis positioning the skin surface a distance from the penetration end, the endcap comprising a hole through which the penetration end passes to puncture the skin;
a force registering member connected to the endcap that registers the amount of force applied to the endcap, the force registering member activating the drive member when a predetermined amount of force is applied to the endcap; and
a penetration end adjustment mechanism and an endcap adjustment mechanism for adjusting the puncture depth of the penetration end of the lancet.

28. A method for puncturing skin to obtain a blood sample with a lancing mechanism, the lancing mechanism including a slot cam, lancet, endcap, drive member, and trigger, the method comprising the acts of:
pressing the user's skin against the endcap connected to a force registering member;
determining the force applied to the endcap; and
initiating the drive member if a predetermined amount of force is applied to the endcap, the drive member causing the slot cam to move in a linear direction, the linear movement of the slot cam causing the lancet to move from a first position to a second position and back to the first position, resulting in a single puncture in the user's skin.

29. The method of claim 28, further including the act of adjusting the position of the penetration end and the endcap to ensure the desired clearance between the skin surface and the penetration end.
